(19) United States
(12) Patent Application Publication
(10) Pub. No.: US 2005/0154259 A1
(43) Pub. Date: Jul. 14, 2005

(54) Magnetically guided colonoscope

(76) Inventor: Thomas J. Demarco, Marietta, GA (US)

Correspondence Address: John D. Daniels, Esq., 511 Foot Hills Road, Higganum, CT, 06441, US

(21) Appl. No.: 11/033,002

(22) Filed: Jan. 11, 2005

Related U.S. Application Data
(60) Provisional application No. 60/535,995, filed on Jan. 14, 2004.

(51) Int. Cl. A61B 1/00

(52) U.S. Cl. 600/114

(57) ABSTRACT

A magnetically guidable medical device for use within the large intestine of a human body. A magnetically guidable guide wire is introduced into the rectum of a body of a patient and maneuvered through the colon under the influence of a controlled magnetic field to a desired location within the body of the patient. An external magnetic field applying means applies an external magnetic field effective to guide the magnetically guidable member within the body of the patient. A colonoscope is advanced over the guide wire so that the advancement and placement of the colonoscope is aided by the magnetically guided guide wire. Either or both the magnetically guidable guide wire and the colonoscope can be viewed using a fluoroscope to assist in positioning it within the body of the patient.

### CROSS REFERENCE TO RELATED APPLICATIONS

This is the Utility Patent application of a provisional patent application Ser. No. 60/535,995, entitled Magnetically Guided Colonoscope, filed Jan. 14, 2004.

### BACKGROUND OF THE INVENTION

The present invention pertains to a magnetically guidable intubation device. More particularly, the present invention pertains to a magnetically guidable intubation device for use in performing medical procedures to an interior of a body; and specifically to performing medical procedures to the large intestine of a human patient.

Applicant is the inventor of U.S. Pat. No. 5,353,807 issued, Oct. 11, 1994, entitled Magnetically Guidable Intubation Device. In accordance with this invention, a magnetically guidable intubation device is provided including performing means for performing a medical procedure. A magnetically guidable member is connected with a device for performing a medical procedure within the body. The magnetically guidable intubation device is effective to be introduced into a patient's body and magnetically guided within the body by an externally applied magnetic field. A flexible retrieving member may be fixedly attached to the magnetically guidable member and is used for retrieving the magnetically guided member from the body. An external magnetic field is applied and is effective to guide the magnetically guidable member within the body to perform the medical procedure. The magnetically guidable member may include a core and a conductive winding wound around the core. A remotely controllable current source may be provided for applying an electric current to the winding, which is effective to induce a magnetic field around the magnetically guidable member. Thus, by changing the field strength and polarity of the magnetic field around the magnetically guidable member, precise control of the magnetically guidable member can be realized.

Conventionally, there are known devices for performing medical procedures within the large intestine of a human patient. A typical device, known as a colonoscope or endoscope, includes a head disposed at a distal end of an elongated flexible member. The head is inserted into the large intestine of a patient through the rectum and is moved forward through the colon. By manually pushing the elongated flexible member forward, the head is moved through the colon.

The head may be equipped with a diagnostic or therapeutic device. For example, a fiber optic imaging device may be used diagnostically by a doctor for viewing the interior of the large intestine to allow visual inspection for polyps or other malformations of the intestinal wall. The head may also carry a therapeutic tool, such as a remotely controlled cauterizing loop mechanism which is capable of removing, a polyp from the mucosa of the large intestine.

The elongated flexible member typically has a hollow interior, which is in communication with an interior chamber of the head. The head holds the therapeutic or diagnostic device. Thus in the case of the fiber optic imaging device, the end of a fiber optic cable bundle and a light source may be contained in the chamber in the head and the fiber optic cable and a power cable for the light source may be enclosed within the flexible elongated member. In the case of the therapeutic device, for example, the cauterizing loop mechanism (known as a polypectomy snare), a cable may be inserted through an operative channel contained within the flexible elongated member so that the cauterizing loop mechanism may be remotely manipulated. At the end of the flexible elongated member opposite the head, an eyepiece may be disposed for viewing the interior of the large intestine through the fiber optic cable bundle. Further, a control device may be disposed at the end for manually manipulating, for example, the cauterizing loop mechanism.

In accordance with the conventional art, the elongated flexible member is relatively stiff so that it may effectively push the head forward through the large intestine. Since the large intestine is not straight, but rather has a number of turns to it, it has been difficult to provide an elongated flexible member, which has both the stiffness necessary for pushing the head forward through the large intestine and the flexibility to maneuver the head around the various curves of the large intestine. During the procedure, the conventional colonoscope has a tendency to bend into what are known as an alpha loop, N-loop an/or reverse alpha loop. These loops in the colonoscope usually occur in sigmoid colon and further advancement of the colonoscope, if the procedure is not abandoned altogether, causes great discomfort due to stretching of the colon. Also, in the conventional method, there is the danger of possible complication of colon perforation due to these loops.

### SUMMARY OF THE INVENTION

It is an object of the present invention to provide an improved means for enabling a colonoscope to be introduced into and guided through colon of the body of a patient.

In accordance with the present invention, a magnetically guidable medical device is provided for use within the large intestine of a human body. A magnetically guidable guide wire is provided. The magnetically guidable guide wire is capable of being introduced into the rectum of a body of a patient and maneuvered through the colon under the influence of a controlled magnetic field to a desired location within the body of the patient. An external magnetic field applying means applies an external magnetic field effective to guide the magnetically guidable member within the body of the patient. A colonoscope advanceable over the guide wire is provided whereby the magnetically guided guide wire aids the advancement and placement of the colonoscope.

The magnetically guidable medical device may further comprise viewing means for viewing the magnetically guidable guide wire and the colonoscope to assist in positioning the guide wire and/or the colonoscope within the body of the patient. The viewing means may be a fluoroscope, MRI, or other means for viewing the interior of a body.

The present invention also pertains to a method of performing a colonoscopy. A magnetically guidable guidewire is passed into the rectum of the body of a patient. The magnetically guidable guidewire is advanced through the colon of the patient to a desired location, such as the cecum. The guidewire is advanced under at least the partial control of a variable magnetic field controllably generated outside the body of the patient. Using the guidewire for directional assistance, a colonoscope is advanced over the guidewire to the desired location. Either or both the magnetically guidable guide wire and the colonoscope can be viewed using a fluoroscope to assist in positioning it within the body of the patient.

### BRIEF DESCRIPTION OF THE DRAWINGS

FIG. 1 is a block diagram and schematic view of the elements of the inventive magnetically guidable medical device;

FIG. 2 is schematic view of the magnetically guidable guide wire inserted through the rectum of the patient and into the colon;

FIG. 3 is a schematic view of the guide wire being advanced through the colon of the patient under the control of an externally applied magnetic field;

FIG. 4 is a schematic view of the guide wire having its magnetic head positioned at the cecum of the colon;

FIG. 5 is a schematic view of the colonoscope inserted through the rectum and passing over the guide wire;

FIG. 6 is a schematic view of the colonoscope being advanced through the colon of the patient with directional guidance from the guide wire; and

FIG. 7 is a schematic view of the colonsocope having its therapeutic head positioned at the cecum of the colon.

### DETAILED DESCRIPTION OF THE INVENTION

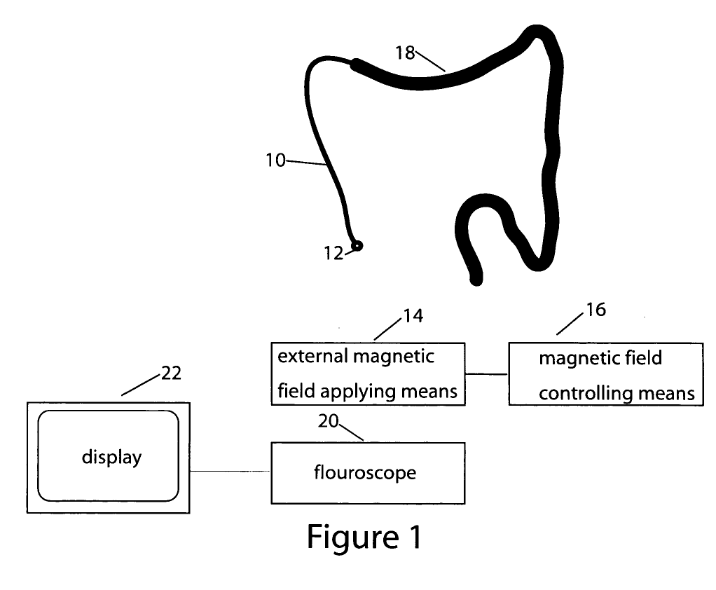

FIG. 1 is a block diagram and schematic view of the elements of the inventive magnetically guidable medical device. The inventive magnetically guidable medical device includes a magnetically guidable guide wire 10 having a magnetic tip 12. The magnetically guidable guide wire 10 is introduced into the rectum of a body of a patient and maneuvered through the colon (not shown) under the influence of a controlled magnetic field. The controlled magnetic field is generated by external magnetic field applying means 16 under the control of magnetic field controlling means 16. A human operator, such as a doctor, may manually operate the magnetic field controlling means 16. Alternatively, or in addition, the magnetic field controlling means 16 can be controlled from an external source, such as an Internet, modem, computer or wireless connection to enable remote and/or computer controlled maneuvering of the magnetically guidable guide wire 10. By controlling the placement of the magnetic tip 12 to a desired location within the body of the patient, the magnetically guidable guide wire 10 creates a pathway for assisting of the advancement of the colonoscope within the body of the patient. The external magnetic field applying means 14 applies the external magnetic field to guide the magnetically guidable guide wire within the body of the patient. As described in more detail below, a colonoscope 18 can then be advanced over the guide wire 10 so that the advancement and placement of the colonoscope 18 is aided by the magnetically guided guide wire 10.

The magnetically guidable medical device may further comprise viewing means for viewing the magnetically guidable guide wire 10 and the colonoscope 18 to assist in positioning the guide wire 10 and/or the colonoscope 18 within the body of the patient. The viewing means may be a video display 22 that displays images from a fluoroscope 20, MRI, or other means for viewing the interior of the body of the patient.

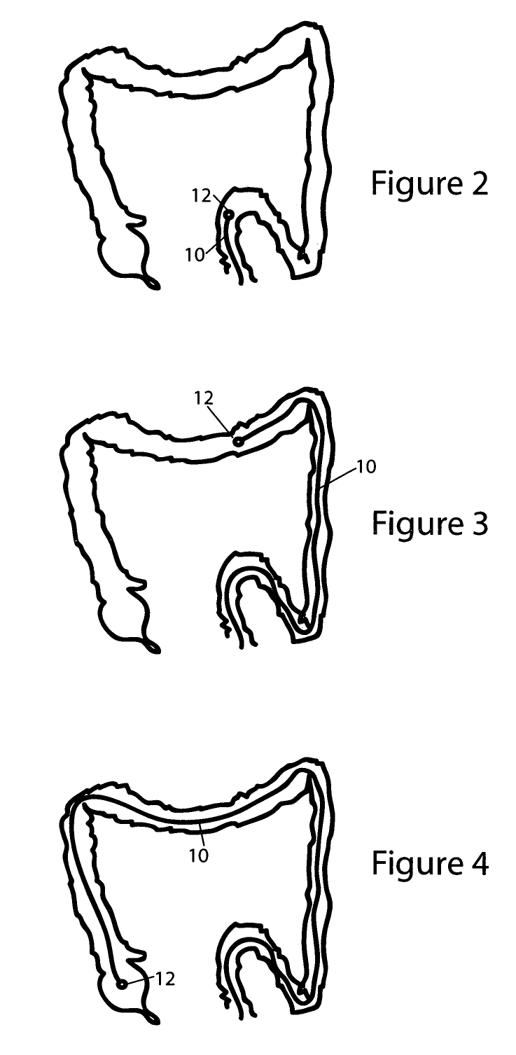

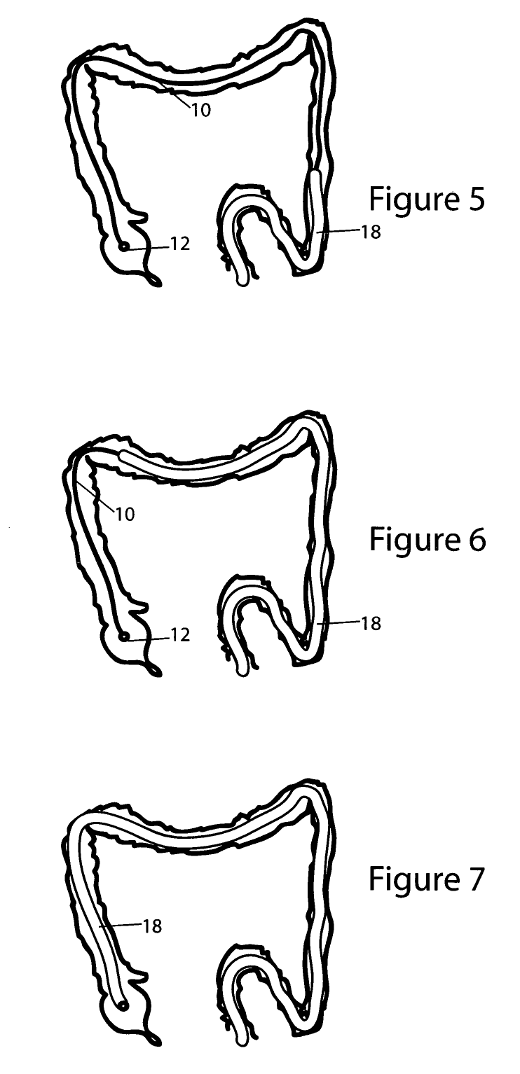

The present invention also pertains to a method of performing a colonoscopy. FIG. 2 is schematic view of the magnetically guidable guide wire 10 inserted through the rectum of the patient and into the colon. In accordance with the inventive method, the magnetically guidable guidewire 10 is passed into the rectum of the body of a patient. FIG. 3 is a schematic view of the guide wire being advanced through the colon of the patient under the control of an externally applied magnetic field. As shown in FIG. 3, the magnetically guidable guidewire 10 is advanced through the colon, being maneuvered through the twists and turns of the large intestine under the control of the externally applied magnetic field. FIG. 4 is a schematic view of the guide wire having its magnetic tip 12 positioned at the cecum of the colon. The magnetic tip 12 drags the guidewire 10 within the body of the patient to a desired location, such as the cecum. The guidewire 10 is advanced under the control of a variable magnetic field controllably generated outside the body of the patient creating a pathway for advancing the colonoscope 18 so that the usual manual manipulation of the colonoscope 14 can be reduced or avoided. Next, as shown in FIG. 5, the colonoscope 18 is inserted through the rectum and passed over the guide wire 10. As shown in FIG. 6, using the guidewire 10 for directional assistance, the colonoscope 18 is advanced over the guidewire 10 to the desired location (shown in FIG. 7). Either or both the magnetically guidable guide wire 10 and the colonoscope can be viewed using a fluoroscope to assist in positioning it within the body of the patient.

During use of the inventive magnetically guidable medical device, the patient, after completing a colon prep, lies on the left side within the confines of a magnetic navigation system, such as the Stereotaxis Niobe magnetic navigation system. The magnetically tipped guidewire 10 is threaded through the biopsy channel of a conventional video colonoscope 18. A section of the guidewire 10 extends from the tip of the colonoscope. The guidewire 10 is passed into the patient's rectum and advanced to the cecum. The guidewire 10 is advanced using the magnetic navigation system. Guidance can be assisted by a fluoroscopy system, such as a digital fluoroscope 20 and display 22. Once the guidewire 10 is in place in the cecum, the video colonoscope 18 is passed over the guidewire 10. The colonoscope 18 may be advanced under direct vision via the video endoscopic system 20, 22. When the proximal colon is reached, the guidewire 10 can be removed. The colonoscope is then gradually withdrawn back to the rectum, proceeding in the conventional fashion, looking for and removing polyps, etc.

In accordance with the present invention, the colonoscopy procedure can be performed with less pain and discomfort to the patient. The patient may not have to by sedated as heavily, if at all, enabling a safer, more convenient colonoscopy procedure.

With respect to the above description, it is realized that the optimum dimensional relationships for parts of the invention, including variations in size, materials, shape, form, function, and manner of operation, assembly and use, are deemed readily apparent and obvious to one skilled in the art. All equivalent relationships to those illustrated in the drawings and described in the specification are intended to be encompassed by the present invention. Therefore, the foregoing is considered as illustrative only of the principles of the invention. Further, since numerous modifications and changes will readily occur to those skilled in the art, it is not desired to limit the invention to the exact construction and operation shown and described. Accordingly, all suitable modifications and equivalents may be resorted to, falling within the scope of the invention.

1. A magnetically guidable medical device for use within the large intestine of a human body comprising: a magnetically guidable guide wire capable of being introduced into the rectum of a body of a patient and maneuvered through the colon under the influence of a controlled magnetic field to a desired location within the body of the patient; an external magnetic field applying means for applying an external magnetic field effective to guide the magnetically guidable member within the body of the patient; and a colonoscope advanceable over the guide wire whereby the advancement and placement of the colonoscope is aided by the magnetically guided guide wire.

2. A magnetically guidable medical device according to claim 1; further comprising viewing means for viewing at least one of the magnetically guidable guide wire and the colonoscope to assist in positioning within the body of the patient.

3. A magnetically guidable medical device according to claim 2; wherein the viewing means comprises a fluoroscope.

4. A method of performing a colonoscopy, comprising the steps of: passing a magnetically guidable guidewire into the rectum of the body of a patient; advancing the magnetically guidable guidewire through the colon of the patient to a desired location, the guidewire being advanced under at least the partial control of a variable magnetic field controllably generated outside the body of the patient; and using the guidewire for directional assistance, advancing a colonoscope over the guidewire to the desired location.

5. A method of performing a colonoscopy according to claim 4, further comprising the step of; viewing at least one of the magnetically guidable guide wire and the colonoscope to assist in positioning within the body of the patient.

6. A method of performing a colonoscopy according to claim 5; wherein the viewing means comprises a fluoroscope.

7. A magnetically guidable medical device for use within the large intestine of a human body comprising: a magnetically guidable guide wire capable of being introduced into the the body of a patient, said guide wire being manuverable through the body under the influence of a controlled magnetic field to a desired location within the body of the patient; an external magnetic field applying means for applying an external magnetic field effective to guide the magnetically guidable member within the body of the patient; and a medical procedure performing instrument advanceable over the guide wire whereby the advancement and placement of the medical procedure performing instrument is aided by the magnetically guided guide wire.

8. A magnetically guidable medical device according to claim 7; further comprising viewing means for viewing at least one of the magnetically guidable guide wire and the medical procedure performing instrument to assist in positioning within the body of the patient.

9. A magnetically guidable medical device according to claim 8; wherein the viewing means comprises a fluoroscope.

10. A magnetically guidable medical device according to claim 7; wherein the medical procedure performing instrument is a colonscope.
